(19) United States
(12) Patent Application Publication
(10) Pub. No.: US 2019/0366032 A1
(43) Pub. Date: Dec. 5, 2019

(54) Method and System for Generating and Providing Notifications for a Circadian Shift Protocol

(71) Applicant: Timeshifter, Inc., Southampton, NY (US)

(72) Inventors: Steven Lockley, Southampton, NY (US); Tony Hanna, Southampton, NY (US); Jacob Ravn, Southampton, NY (US); Mickey Beyer-Clausen, Southampton, NY (US); Fredric Maxik, Cocoa Beach, FL (US)

(73) Assignee: Timeshifter, Inc., Southampton, NY (US)

(21) Appl. No.: 16/432,544

(22) Filed: Jun. 5, 2019

Related U.S. Application Data
(60) Provisional application No. 62/680,887, filed on Jun. 5, 2018.

(51) Int. Cl. A61M 21/02

(52) U.S. Cl. CPC A61M 21/02; A61M 2021/0044

(57) ABSTRACT

A method of determining and presenting a protocol for adjusting a circadian rhythm of a user comprising receiving an indication of an intended circadian shift comprising a shift magnitude, receiving an indication of a user circadian phase, defining a user circadian shift protocol responsive to the shift magnitude and the indication of the user circadian phase, the user circadian shift protocol comprising one or more activities scheduled to be performed at a certain date and time, the activities comprising at least one of a light exposure or light avoidance activity, a chronobiotic activity, a nutritional consumption activity, a physical activity, and a rest activity. The method may further provide an indication to perform each activity on a user device.

### RELATED APPLICATIONS

This application claims priority under 35 U.S.C. § 119(e) of U.S. Provisional Patent Application Ser. No. 62/680,887 (Attorney Docket No. 3788.00001) filed on Jun. 5, 2018 and titled METHOD AND SYSTEM FOR GENERATING AND PROVIDING NOTIFICATIONS FOR A CIRCADIAN SHIFT PROTOCOL. The content of this application is incorporated herein by reference.

### FIELD OF THE INVENTION

The present invention relates to systems and methods for creating and implementing circadian shift protocols responsive to user inputs and external circumstances known or unknown to the user and providing notifications to the user for the same.

### BACKGROUND OF THE INVENTION

Challenges of shifting an individual's circadian rhythm are well known in the art. Current solutions to shifting circadian rhythms generate protocols that fail to consider the circadian phase of the individual, do not consider whether what is indicated by the protocol is practical and likely to be adhered to be the individual, and are not adapted to prepare multiple, successive protocols that may conflict with one another, as well as other deficiencies. Accordingly, there is a need in the art for a circadian shifting protocol method and system for generating the same that addresses these deficiencies.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to a method of determining and presenting a protocol for adjusting a circadian rhythm of a user comprising receiving an indication of an intended circadian shift comprising a shift magnitude, receiving an indication of a user circadian phase, and defining a user circadian shift protocol responsive to the shift magnitude and the indication of the user circadian phase, the user circadian shift protocol comprising one or more activities scheduled to be performed at a certain date and time. The activities may comprise at least one of a light exposure or light avoidance activity, a chronobiotic activity, a nutritional consumption activity, a physical activity, and a rest activity. The method may further comprise providing an indication to perform each activity on a user device. Each of the activities are indicated to the user to affect the intended circadian shift.

In some embodiments, the method may further comprise receiving a sleep pattern for the user, wherein the user circadian shift protocol is defined responsive to the sleep pattern. In some embodiments, the method may further comprise receiving an activity preference indication from the user identifying an activity the user will not perform, defining an excluded activity; wherein defining the user circadian shift protocol comprises defining one or more activities excluding the excluded activity.

In some embodiments, the method may further comprise receiving an indication from a user of non-performance of an activity, defining a non-performed activity and adjusting the user circadian shift protocol responsive to the non-performed activity. In some embodiments, the method may further comprise identifying a first resting activity comprised by the user circadian shift protocol and a second resting activity comprised by the user circadian shift protocol, where no other resting activities comprised by the user circadian shift protocols are scheduled between the first resting activity and the second resting activity, determining a length of time between the first resting activity and the second resting activity, defining a resting gap, and determining if the resting gap exceeds a maximum resting gap threshold. Upon determining the resting gap exceeds the maximum resting gap threshold, modifying the user circadian shift protocol by at least one of altering at least one of the first resting activity and the second resting activity such that the resting gap is reduced to no longer exceed the maximum resting gap threshold and creating a new resting activity scheduled between the first resting activity and the second resting activity.

In some embodiments, the method may further comprise receiving an indication of an intervening user event, determining whether the intervening user event conflicts with the performance of an activity, and modifying the user circadian shift protocol responsive to determining the intervening user event conflicts with the performance of an activity. In some embodiments, the method may further comprise identifying an activity comprised by the user circadian shift protocol that is concurrent with or in the proximate future of the current time, defining an identified action, receiving an indication of a present location of the user, identifying a location proximate to the present location of the user that can facilitate performance of the identified action, defining an activity location, and providing an indication of the existence and location of the activity location on the user device.

In some embodiments, the method may further comprise receiving an indication of a present circadian rhythm status of the user, receiving an indication of a present location of the user, determining a preferred circadian rhythm status responsive to the present location of the user, and determining if the present circadian rhythm of the user is mismatched with the preferred circadian rhythm status. The user circadian shift protocol is defined responsive to the present circadian rhythm of the user is mismatched with the preferred circadian rhythm status.

In some embodiments, the method may further comprise determining whether an advancing circadian shift protocol that advances a circadian rhythm of the user or a delaying circadian shift protocol that delays the circadian rhythm of the user is preferable, defining a preferred shifting direction; wherein the user circadian shift protocol is defined responsive to the preferred shifting direction. In some embodiments, the method may further comprise determining a present circadian rhythm status of the user responsive to the current time and the activities scheduled prior to the current time and sending the present circadian rhythm status to a remote computerized device.

In some embodiments, the user circadian shift protocol is a first user circadian shift protocol and the method may further comprise receiving a second indication of an intended circadian shift comprising a second shift magnitude, and defining a second user circadian shift protocol responsive to the second shift magnitude, the second user circadian shift protocol comprising one or more activities scheduled to be performed at a certain date and time. The activities may comprise at least one of a light exposure or light avoidance activity, a chronobiotic activity, a nutritional consumption activity, a physical activity, and a rest activity. The method may further comprise determining whether an activity of the first user circadian shift protocol conflicts with an activity of the second user circadian shift protocol. Upon determining an activity of the first user circadian shift protocol conflicts with an activity of the second user circadian shift protocol, the method may further comprise modifying at least one of the first user circadian shift protocol and the second user circadian shift protocol to remove the conflict and providing an indication to perform each activity of each of the first user circadian protocol and the second user circadian protocol on the user device.

In some embodiments, the method may further comprise sending a command to a device responsive to an activity comprised by the user circadian shift protocol. In further embodiments, the command is selected from the group consisting of altering display characteristics of a display, causing a display of information on a display, altering light emission characteristics of a lighting device, altering a status of a window-related device to alter ambient light conditions, initiating production of a beverage comprising a stimulant, and transmitting a message to a person regarding provision of at least one of a chronobitotic and nutrition.

In some embodiments, the indication of the intended circadian shift may be related to travel information for a user trip, the travel information comprising a departure date, a departure time, a departure location, an arrival date, an arrival time, and an arrival location; and wherein a time zone difference between the departure location and the arrival location defines the shift magnitude. Furthermore, the travel information may further comprises a stopover start time and a stopover end time, the time between the stopover start time and the stopover end time defines a stopover duration. The method may further comprise determining if the stopover duration is sufficient for an activity and, upon determining the stopover duration is sufficient for an activity, defining the user circadian shift protocol to comprise an activity during the stopover duration, defining a stopover activity. The stopover activity may be selected from the activities consisting of a light exposure or light avoidance activity, a chronobiotic activity, a nutritional consumption activity, a physical activity, and a rest activity.

Additionally, the user circadian shift protocol may comprise a maximum daily shift, and the method may further comprise determining a pre-arrival time defined as a length of time between a present time and the arrival time on the arrival date, determining a pre-arrival required daily shift to accomplish a complete circadian shift to a time zone associated with the arrival location, determining whether the pre-arrival required daily shift exceeds the maximum daily shift, and upon determining the pre-arrival required daily shift exceeds the maximum daily shift, modifying the user circadian shift protocol to accomplish a partial circadian shift by the arrival time on the arrival date.

In some embodiments, the method may further comprise receiving travel information for an individual other than the user, defining a companion, comprising a companion departure date, a companion departure time, a companion departure location, a companion arrival date, a companion arrival time, and a companion arrival location, determining a companion time zone difference between the companion departure location and the companion arrival location, defining a companion shift magnitude, and receiving personal information for each of the user and the companion. The user circadian shift protocol may be determined responsive to the personal information of at least one of the user and the companion. Furthermore, the method may further comprise receiving travel information for the companion comprising a companion departure date, a companion departure time, a companion departure location, a companion arrival date, a companion arrival time, and a companion arrival location, determining a companion time zone difference between the companion departure location and the companion arrival location, defining a companion shift magnitude, and defining a companion circadian shift protocol responsive to the shift magnitude, the circadian shift protocol comprising one or more activities. The activities may comprise at least one of a light exposure or light avoidance activity, a chronobiotic activity, a nutritional consumption activity, a physical activity, and a rest activity. The method may further comprise providing an indication to perform each activity of the companion circadian shift protocol on a companion device.

In some embodiments, the method may further comprise receiving information about a change in at least one of the departure time and the departure date, defining travel change information and adjusting the user circadian shift protocol responsive to the travel change information. In some embodiments, the user circadian shift protocol is a first user circadian shift protocol, and wherein the travel information further comprises a second departure date, a second departure time, a second departure location, a second arrival date, a second arrival time, and a second arrival location. The method may further comprise determining a time zone difference between the second departure location and the second arrival location, defining a second shift magnitude and defining a second user circadian shift protocol responsive to the second shift magnitude, the second user circadian shift protocol comprising one or more second activities. The second activities may comprise at least one of a light exposure or light avoidance activity, a chronobiotic activity, a nutritional consumption activity, a physical activity, and a rest activity. The method may further comprise providing an indication on the user device to perform each activity of the second user circadian shift protocol. Furthermore, the method may comprise determining whether the second user circadian shift protocol overlaps with the first circadian shift protocol. Upon determining the second user circadian shift protocol overlaps with the first circadian shift protocol, the method may further comprise modifying the first user circadian shift protocol to accomplish a partial circadian shift and modifying the second user circadian shift protocol to reflect the first user circadian shift protocol accomplishing a partial circadian shift.

In some embodiments, the user circadian shift protocol comprises an activity scheduled to be performed after the departure time and before the arrival time.

Additionally, embodiments of the invention may be directed to a method of determining and presenting a protocol for adjusting a circadian rhythm of a user comprising receiving an indication of an intended circadian shift comprising a shift magnitude, receiving a user circadian phase, receiving an activity preference indication from the user identifying an activity the user will not perform, defining an excluded activity, receiving an indication of a present circadian phase status of the user, receiving an indication of a present location of the user, determining a preferred circadian phase status responsive to the present location of the user, and determining if the present circadian phase of the user is mismatched with the preferred circadian phase status. The method may further comprise determining whether an advancing circadian shift protocol that advances a circadian rhythm of the user or a delaying circadian shift protocol that delays the circadian rhythm of the user is preferable, defining a preferred shifting direction and defining a user circadian shift protocol responsive to the shift magnitude, the chronotype preference, the preferred shifting direction, and if the present circadian phase of the user is mismatched with the preferred circadian phase status, the user circadian shift protocol comprising one or more activities scheduled to be performed at a certain date and time excluding the excluded activity. The activities may comprise a first rest activity, a second rest activity, and at least one of a light exposure or light avoidance activity, a chronobiotic activity, a nutritional consumption activity, and a physical activity. The method may further comprise providing an indication to perform each activity of the user circadian shift protocol on a user device and sending a command to a device responsive to an activity comprised by the user circadian shift protocol. Each of the activities are presented to the user to affect the intended circadian shift. A length of time between the first resting activity and the second resting activity do not exceed a maximum resting gap.

Additionally, embodiments of the invention may be directed to a method of determining and presenting a protocol for adjusting a circadian rhythm of a user comprising receiving a travel information related to an upcoming user trip comprising a departure date, a departure time, a departure location, an arrival date, an arrival time, and an arrival location, a stopover start time and a stopover end time, wherein a time zone difference between the departure location and the arrival location defines a shift magnitude, and wherein the time between the stopover start time and the stopover end time defines a stopover duration, determining if the stopover duration is sufficient for an activity, receiving a chronotype preference for the user, and defining a user circadian shift protocol responsive to the shift magnitude, the chronotype preference, and if the stopover duration is sufficient for an activity, the user circadian shift protocol comprising one or more activities scheduled to be performed at a certain date and time. The activities may comprise at least one of a light exposure or light avoidance activity, a chronobiotic activity, a nutritional consumption activity, a physical activity, and a rest activity. The method may further comprise providing an indication to perform each activity of the user circadian shift protocol on a user device. Each of the activities are presented to the user to affect a circadian shift. The user circadian shift protocol comprises an activity during the stopover duration if the stopover duration is sufficient for an activity selected from the activities consisting of a light exposure or light avoidance activity, a chronobiotic activity, a nutritional consumption activity, a physical activity, and a rest activity.

### BRIEF DESCRIPTION OF THE DRAWINGS

Some embodiments of the present invention are illustrated as an example and are not limited by the figures of the accompanying drawings, in which like references may indicate similar elements.

FIG. 1 is a flowchart illustrating a method of generating and displaying a circadian shift protocol according to an embodiment of the invention.

FIG. 2 is a flowchart illustrating a method of ensuring a circadian shift protocol includes adequate resting activities according to an embodiment of the invention.

FIG. 3 is a flowchart illustrating a method of modifying a circadian shift protocol responsive to an intervening event according to an embodiment of the invention.

FIG. 4 is a flowchart illustrating a method of facilitating performance of an activity of a circadian shift protocol according to an embodiment of the invention.

FIG. 5 is a flowchart illustrating a method of defining a circadian shift protocol responsive to a circadian status of the according to an embodiment of the invention.

FIG. 6 is a flowchart illustrating a method of providing an indication of a user circadian status according to an embodiment of the invention.

FIG. 7 is a flowchart illustrating a method of generating a second circadian shift protocol and modifying a circadian shift protocol responsive to a shift protocol conflict according to an embodiment of the invention.

FIG. 8 is a flowchart illustrating a method of defining a circadian shift protocol comprising an activity to be performed during a travel stopover according to an embodiment of the invention.

FIG. 9 is a flowchart illustrating a method of defining a partial circadian shift protocol according to an embodiment of the invention.

FIG. 10 is a flowchart illustrating a method of defining a circadian shift protocol for a travel companion according to an embodiment of the invention.

FIG. 11 is a flowchart illustrating a method of defining a circadian shift protocol for two individuals according to an embodiment of the invention.

FIG. 12 is a flowchart illustrating a method of modifying a circadian shift protocol responsive to a travel delay according to an embodiment of the invention.

FIG. 13 is a flowchart illustrating a method of defining and displaying a second circadian shift protocol subsequent to a first circadian shift protocol according to an embodiment of the invention.

FIG. 14 is a flowchart illustrating a method of modifying the circadian shift protocols of FIG. 13 responsive to a conflict there between according to an embodiment of the invention.

FIGS. 15-41 are illustrative graphics user interfaces of a method of receiving information for, defining, and displaying a circadian shift protocol according to embodiments of the invention.

FIG. 42 is a schematic depiction of connected devices that may be part of the present invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

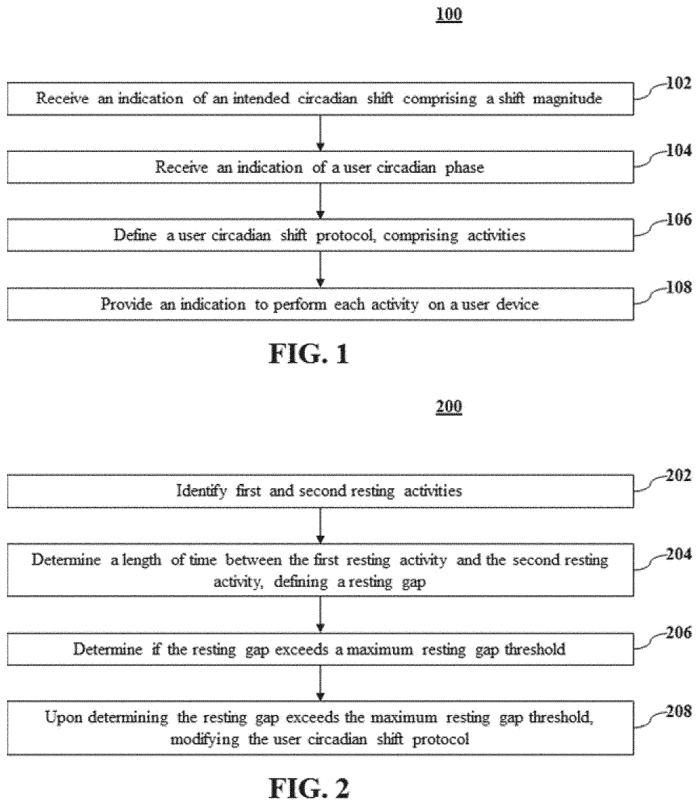

Referring now to FIGS. 1-14, a computer-implemented method of identifying circadian rhythm change scenarios, determining a circadian rhythm adjustment program responsive to the identified circadian rhythm change scenario, and prescribing at least one intervention designed to effect a change to a circadian rhythm of a circadian rhythm for an individual associated with the identified circadian rhythm change scenario. The method may be implemented on a device comprising a display, a processor operably coupled to the display and operable to control both the display content and the brightness of the display, a memory, and, in some embodiments, a network communication device, the network communication device being operable to communicated using at least one communication protocol, including, but not limited to, wireless communication protocols such as 802.XX protocols, such as Wi-Fi, Bluetooth, Zigbee, Z-Wave, and the like. Additionally, the network communication device may be operable to communicated across at least one of a Personal Area Network (PAN), a Local Area Network (LAN), or a Wide Area Network (WAN), such as the Internet. The memory may comprise thereon a computer program comprising instructions that are executable by the processor so as to perform the following method. In some embodiments, the device may be a smartphone, comprising a touchscreen display, however it is contemplated and included within the scope of the invention that the method may be performed by any computerized device as described above and operable to receive user input, including input performed using a mouse, a keyboard, a touchscreen, a sensor, and any other user input device as is known in the art. Steps of receiving information may be performed by one of a user input device and the network communication device. Steps of determining, identifying, selecting, creating, adjusting a circadian shift protocol, altering a circadian shift protocol, and modifying a circadian shift protocol is performed by the processor. The steps of displaying and providing an indication may be performed by the processor in conjunction with the display device. Steps of transmitting a command to a computerized device and, in some embodiments, receiving user inputs may be performed by the network communication device. In other embodiments, the method may be implemented on a server that is in communication with a user device via the Internet, such that the following are shown on a display of the user device, user inputs and external circumstances are received by the user device and transmitted to the server, which processes said user input in the generation of the circadian shift protocol. Such a server may further be in communication with the user device to provide notifications according to a determined circadian shift protocol, as will be described in detail below.

Referring now to FIG. 1, a flowchart illustrating a method 100 of determining and presenting a protocol for adjusting a circadian rhythm of a user is presented. The method 100 may comprise receiving an indication of an intended circadian shift for a user, comprising a shift magnitude 102. The circadian shift may be understood as changing the circadian rhythm of the user such that the user's waking/sleeping diurnal pattern is changed to different hours, i.e. the user wakes up at a different time and goes to sleep at a different time. The intended circadian shift represents a target or desired circadian rhythm of the user, taking the form of any target for a circadian rhythm including a target time to wake up, a target time to go to sleep, a target time to be at peak performance, and any other target time for a biological process associated with the circadian rhythm The motivation for such a target circadian rhythm can be varied, including, but not limited to, travelling to a different time zone, desire to rise earlier or later than the user rises at present, going to sleep earlier or later than the user goes to sleep at present, a desire to maximize the body's peak performance, a change in work schedule, and adjustments related to medical conditions, such as insomnia, maximizing drug delivery effectiveness, preparing the body for surgery, and the like. While the portions of the discussion will be constrained to effecting a change in a circadian rhythm as a result of travel, all of the aforementioned motivations and any others as may exist may similarly be presented as options for the user's selection, and follow-up questions pertinent to those objectives may similarly be asked of the user, and input received from the user, to program a circadian rhythm change protocol accordingly.

In some embodiments, the shift magnitude may be expressly provided, i.e. the user indicates a desire to shift their circadian rhythm forward one hour. In some embodiments, the shift magnitude may be understood as a difference between a current, future, or otherwise assumed circadian rhythm of the user and a target circadian rhythm. In some embodiments, the shift magnitude may be determined from information provided by the user, as will be discussed in greater detail herein below.

The indication for the intended circadian shift can take many forms. In some embodiments, the indication can comprise travel information related to a user trip. The travel information may indicate a change in time zone, where the change in time zone at least partially, and in some embodiments fully, defines the shift magnitude. The travel information may comprise a departure date, a departure time, a departure location, an arrival date, an arrival time, and an arrival location. The difference between time zones of the departure location and the arrival location may define the shift magnitude. This type of inferring or determining a shift magnitude is exemplary only and does not limit the scope of the invention. A shift magnitude may be determined or inferred from other information, including, but not limited to, a scheduled event (such as a sporting event, academic evaluation, or meeting, etc.), a prescribed time to take medication, a change in time related to daylight saving time, a change in work schedule (including a change in day/night or first/second/third shift), and the like.

The method may design a protocol responsive to the entered flights, accounting for the duration of stopovers or at any destination, such that, in some instances, a partial circadian shift is affected, such that the user may more expediently shift to a circadian rhythm aligned with the time zone to which they are going, if the magnitude difference in time zones between the travel destination and the return, or onwards, destination is too great for a complete shift to be accomplished, the number of days until departure is too small to affect a complete circadian change so as to adjust the user to a circadian rhythm aligned with the destination time zone, and/or the duration of the trip is so short as to not warrant a complete circadian shift. These motivations, and any others, may each be weighted by the method, in some instances according to user input in terms of user preference, and the circadian shift protocol designed accordingly.

The method 100 may continue with receiving an indication of a user circadian phase 104. The indication of the user circadian phase may take many forms, including, but not limited to, an indication from the user as to what their diurnal preference, or choronotype, is (e.g. early bird, night owl, or neither). Further indications of the user's circadian phase may include physiological measurements. These indications are exemplary only, and any indication of a user circadian phase is contemplated and included within the scope of the invention.

The method 100 may continue with defining a user circadian shift protocol responsive to the shift magnitude and the indication of the user circadian phase. Defining the protocol may comprise scheduling one or more circadian-shifting activities to be performed at a certain date and time, with the totality of the activities defining the protocol. Each of the activities may be designed to affect the intended circadian shift. The types of activities comprised by the protocol may include, but are not limited to, light exposure and/or light avoidance activities, chronobiotic activities, nutritional consumption activities, physical activities, and rest activities. Light exposure/avoidance activities may include, but are not limited to, seeking out exposure to light, seeking out exposure to daylight, seeking out exposure to light comprising light within a blue range of the physical spectrum (i.e. light within a wavelength range from 450 nanometers (nm) to 485 nm), avoiding light, avoiding daylight, and avoiding light comprising light within the blue range, including any timing, duration, pattern, and intensity. Chronobiotic activities may include ingestion of sleep aid, or supplements or chronobiotics (drugs that shift the timing of circadian rhythms(s)) such as melatonin or its agonists (by any method, including melatonin pills, patches, and beverages). Nutritional consumption activities may include ingestion of stimulants such as caffeine (by any method, including drinking caffeinated beverages such as tea or coffee or pills) or other nutritional intake (such as eating meals or snacks). Physical activities may include exercise of any type, and of any duration, intensity, timing, and frequency. Rest activities may include overnight and/or day time sleeping intended to accomplish one or more complete sleep cycles including wake up times and going to sleep times, and napping (i.e. sleep that does not accomplish complete sleep cycles). It is contemplated and included within the scope of the invention that each of these activities may be characterized as “strongly recommended/mandatory,” “optional,” and other indications of the importance of performing the action. Such indications may be responsive to the effect each activity has on shifting the circadian rhythm of the user, with more effective activities tending to be characterized as “strongly recommended/mandatory” and less effective activities tending to be characterized as “optional.”

The method 100 may further comprise providing an indication to perform each activity comprise by the protocol on a user device 106. The indications may comprise a textual reminder to perform the activity, an iconographic reminder to perform the activity, a haptic indication, generating a colored light, an audio indication in the form of a voice reminder, a musical/tonal reminder, or any other sound to indicate the activity to be performed. In some embodiments, the indication may be presented within the context of a calendar, with the portion of the calendar occupied by the indication to perform the activity representing the date and time during which the activity is to be performed. Examples of such indications are shown in FIGS. 34-41, as will be discussed in greater detail below.

In some embodiments, the user may provide an indication as to how they want to receive notifications about the circadian shift protocol resulting from the method. Such notifications may be provided by any means or method known in the industry, including, but not limited to, push notifications, text messages, multimedia messages, e-mails, automated telephone calls, and the like.

In some embodiments, a sleep pattern for the user may also be received, distinct from the indication of the user circadian phase. In such embodiments, the protocol may be defined responsive to the received sleep pattern. In some embodiments, an activity preference may be received from the user. Such activity preferences may include an activity the user does not wish to perform (defining an excluded activity), an activity the user prefers to perform (defining a preferred activity), and time ranges for either of the excluded or preferred activities, indicating if there is just a certain time the user wishes to exclude or promote the respective activities. The protocol may be defined responsive to either or both of the excluded activity and the preferred activity, as well as the respective time ranges.

Resting activities may comprise one or more naps. Information on when and for how long to nap either before, during or after a flight may be provided, either as specific timing or ranges of opportunity during which a shorter nap could be taken. These naps may be further categorized into ‘strongly recommended/mandatory’ or ‘optional’ based on prior sleep opportunity, time awake and/or the likelihood that people will need them or can do them.

Napping, both in terms of time and duration, may have certain controls. For example, a nap after landing may be no longer than three hours in length, and must end no later than two hours after a user's typical sleeping end time. A nap before an early flight is limited to three hours in length and can only start a maximum of two hours before a user's typical sleep start time. Moreover, pre-flight naps may be limited to one hour in length.

In addition to the times when sleep is recommended on the airplane, optional naps may also be indicated when there is an extended episode of time without scheduled sleep on the ground, in order to avoid excessive sleep loss. These will be scheduled to occur at times when they will have the least impact on light exposure or the ability to sleep at the destination.

In some embodiments, an indication of non-performance of an activity may be received, defining a non-performed activity. Such an indication may be for an activity that was intended to have already been performed, intended as being presently performed, or intended to be performed in the future. The protocol may be modified to reflect the non-performed activity while still accomplishing the intended circadian shift.

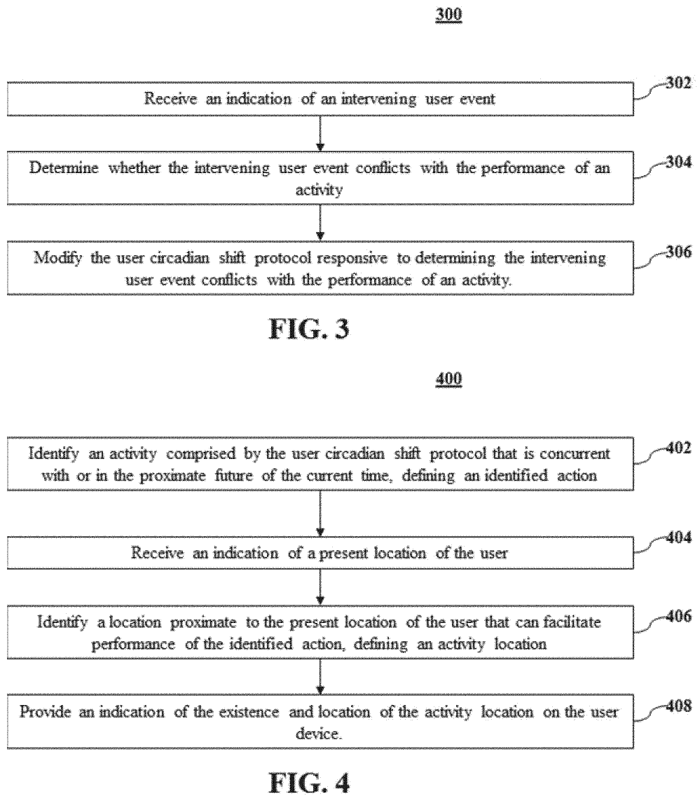

Referring now to FIG. 2, a method 200 of ensuring a circadian shift protocol includes adequate resting activities according to an embodiment of the invention. The method 200 may comprise identifying a first resting activity and a second resting activity of a user circadian shift protocol. The first and second resting activities may be selected such that there are no other resting activities comprised by the protocol are scheduled between them, i.e. they are succeeding resting activities. The method 200 may continue with determining a length of time between the first and second resting activities, defined as a resting gap 204. This may be measured as a stop time of the first resting activity and a start time of the second resting activity. The method 200 may continue with determining if the resting gap exceeds a resting gap threshold 206. The resting gap threshold may be understood as a maximum amount of time that is preferred not to be exceeded. This may also be understood as a maximum continuous waking duration. The resting gap may be pre-defined, and in some embodiments may be defined responsive to a user indication, such as the user's circadian phase or chronotype, or a maximum amount of time selected by the user. Upon determining the resting gap exceeds the maximum resting gap threshold, the protocol may be modified by at least one of altering at least one of the first resting activity and the second resting activity such that the resting gap is reduced to no longer exceed the maximum resting gap threshold, and creating a new resting activity scheduled between the first resting activity and the second resting activity.

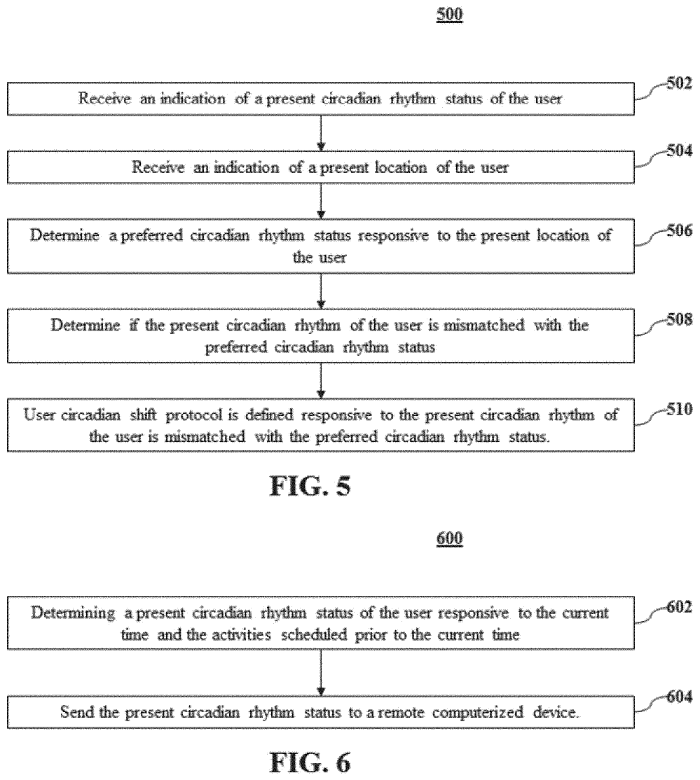

Referring now to FIG. 3, a method 300 of modifying a circadian shift protocol responsive to an intervening event according to an embodiment of the invention is presented. The method 300 may comprise receiving an indication of an intervening user event 302. An intervening user event may be understood as an event related to the user that is not part of the user circadian shift protocol, but is scheduled to occur during the duration of the user circadian shift protocol. The intervening event may be received as an input from the user or other individual, from a calendar (e.g. a calendar provides an indication that an event is occurring at the same time as an activity comprised by the protocol), or any other means or method of receiving an indication of an event. The method 300 may comprise determining whether the intervening user event conflicts with the performance of an activity 304. Such a determination may include first determining if the event coincides with an activity comprised by the protocol, e.g. they are scheduled to occur at the same time. If there is coincidence, the determination may further comprise determining if the nature of the intervening event and the type of activity precludes performance of the activity. For example, if the event is attending a concert and the activity is sleeping, the event precludes performance of the activity. As another example, if the event is to attend a meeting and the activity is to consume caffeine, specifically drink a caffeine-containing beverage, the event does not preclude performance of the activity. If the event precludes performance of the activity, the method 300 may continue with modifying the protocol 306. Such modification may include changing the activity type and/or rescheduling the activity.

Additionally, when determining the circadian shift protocol, certain limitations may be imposed upon when certain activities, exposures, and ingestions occur. For example, users may be required to wake up at least four hours prior to travelling, to permit for adequate transit and check-in time at the airport, which might be in conflict with what a circadian shift would suggest (e.g. sleeping until two hours prior to take-off may be more conducive to obtaining a desired circadian shift). Additionally, as another example, limitations on when a user is unable to comply with certain recommended activities, exposures, and ingestions are contemplated and accounted for. For example, while it might result in a more favorable circadian shift if complied with, an individual may be unable to sleep during a recommended time period, particularly if that individual is boarding a flight or scheduled to travel by car or taxi, for example. Accordingly, the method may take into account such real world or environmental factors, and may generate a circadian shift protocol that eliminates recommendations that are impossible or difficult to comply with, instead making recommendations that have a similar, although moderated, circadian shifting effect. Additionally, as another example, limitations on when a user is likely to comply with certain recommended activities, exposures, and ingestions are contemplated and accounted for. For example, while it might result in a more favorable circadian shift if complied with, an individual may be unlikely to sleep from 4 a.m. to 11 a.m., particularly if that individual has indicated they have a ‘morning type’ diurnal preference, e.g. early bird. Accordingly, the method may comprise creating a circadian shift protocol that has the user sleep from 2 a.m. to 9 a.m. (or some other time period as may be likely to be complied with by the user) and then avoid sunlight from 9 a.m. to 11 a.m. In this way, the method may generate a circadian shift protocol that minimizes recommendations that are unlikely or very difficult to comply with, instead making recommendations that have a similar, although moderated, circadian shifting effect. Additionally, as another example, limitations on when a user is likely to comply with certain recommended activities, exposures, and ingestions are contemplated and accounted for. For example, while it might result in a more favorable circadian shift if complied with, an individual may be not be able to sleep or nap right upon arrival to a destination as the individual will first need to get their luggage, drive to their hotel, check in, unpack their toothbrush etc. before they can go to sleep.

The circadian shift protocol may be connected to a user's calendar, allowing appointments and tasks to be scheduled at a time optimal for such appointment or task. If more users connect their circadian shift protocol to their calendars, the calendar may recommend the best time to meet, talk, or executive tasks together, based on their circadian shift protocols.

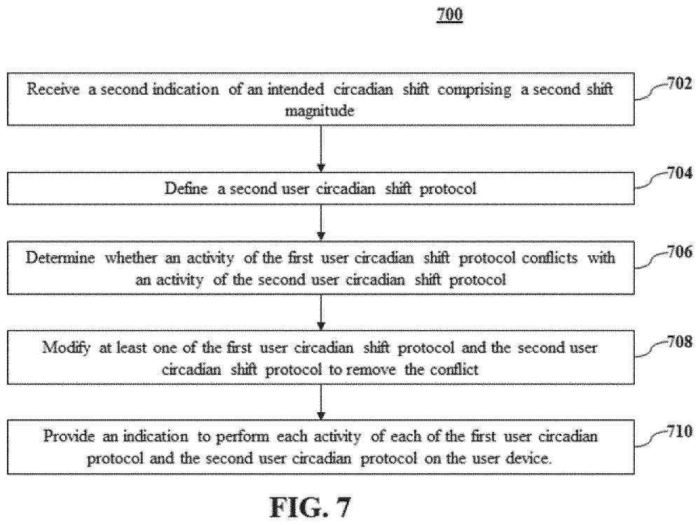

Referring now to FIG. 4, a method 400 of facilitating performance of an activity of a circadian shift protocol according to an embodiment of the invention is presented. The method 400 may comprise identifying an activity comprised by the user circadian shift protocol that is concurrent with or in the proximate future of the current time, defining an identified action 402. The method 400 may continue with receiving an indication of a present location of the user 404. Such an indication may be received by any means or method known in the art, including, but not limited to, receiving a location indication from a GPS device, including a GPS device comprised by a user device, a location indication inferred from an IP address or Wi-Fi network IP address, an indication from a map software operated by the user or other individual associated with the user, and the like. The method 400 may continue with identifying a location proximate to the location of the user that can facilitate performance of the identified action, defining an activity location 406. Such activity locations are responsive to the type of activity. For example, if the activity is a physical activity, specifically exercising, the activity location could be a park with a running trail, a fitness center or gymnasium, and the like. As another example, if the activity is to consume a caffeine-containing beverage, the activity location may be a coffee shop, tea shop, or vending machine containing energy drinks. The identification of the activity location may result from querying a service or database that comprises the location and types of goods or services at such locations with the present location of the user and the identified action, as is known in the art. The method 400 may continue with providing an indication of the existence and location of the activity location on the user device 408. Such an indication may include audio, graphical, and/or textual indications of the activity location, where it is located, navigation information, open-close times, and how it is related to the identified action.

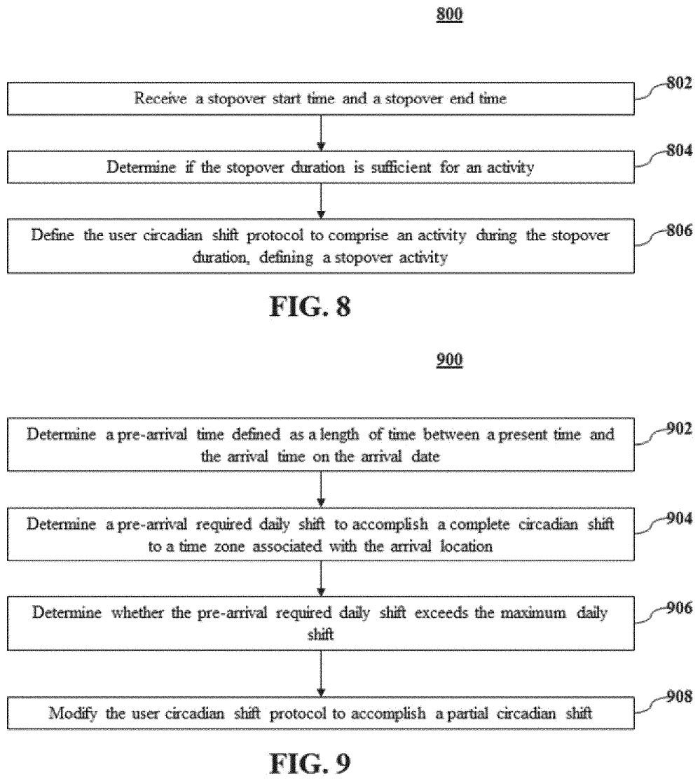

Referring now to FIG. 5, a method 500 of defining a circadian shift protocol responsive to a circadian status of the according to an embodiment of the invention is presented. The method 500 may comprise receiving an indication of a present circadian rhythm status of the user 502. Such an indication may comprise any information that demonstrates, suggests, or otherwise supports a determination of the circadian rhythm status of the user, including, but not limited to, sleep history information, waking time information, time of day, physiological measurement information, and the like. The method 500 may continue with receiving a present location of the user 504, similar to step 404 of FIG. 4 as described herein above. The method 500 may continue with determining a preferred circadian rhythm status for the user responsive to the present location of the user 506. More specifically, a circadian rhythm may be determined for the user based on the user location, and based on one or both of the time of day and the user's circadian phase/chronotype, the preferred circadian rhythm status may be determined. The method 500 may continue with determining if the present circadian rhythm of the user is mismatched with the preferred circadian rhythm status 508. Such a mismatch may be determined by comparing the user's present circadian rhythm and identifying if they are ahead, behind, or aligned with the preferred circadian rhythm status. If there is a mismatch, the method 500 may continue with defining the user circadian shift protocol responsive to the mismatch 510. Defining the protocol responsive to the present circadian rhythm status of the user increases compliance with and effectiveness of the protocol.

In some embodiments, defining the user circadian shift protocol may comprise determining whether advancing the user's circadian rhythm (e.g. making their rise time and bed time earlier) or delaying their circadian rhythm (e.g. making their rise time and bed time later) is most effective and preferable. Such a determination may be made responsive to at least the shift magnitude, the direction of the shift, and the user circadian phase, including the user chronotype. It may further be determined whether an advancing circadian shift protocol that advances the user's circadian rhythm or a delaying circadian shift protocol that delays the user's circadian rhythm is preferred, defining a preferred shifting direction. Accordingly, the user circadian shift protocol may be defined responsive to the preferred shifting direction.

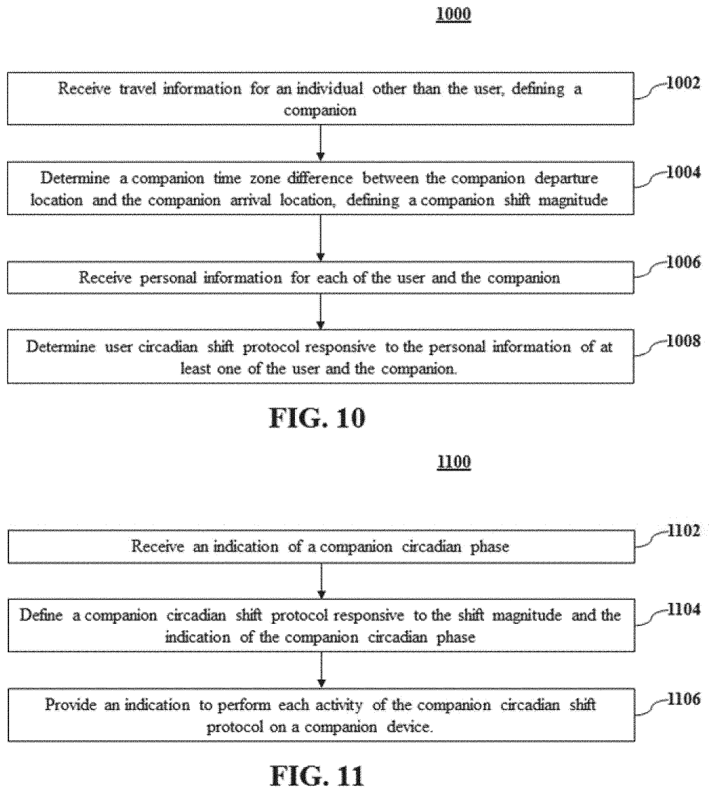

Referring now to FIG. 6, a method 600 of providing an indication of a user circadian status according to an embodiment of the invention is presented. The method 600 may comprise determining a present circadian rhythm status of the user 602. Such a status may be determined responsive to at least the current time and the activities scheduled prior to the current time. This determination may presume the performance of the previously scheduled activities and their attending effect on the circadian rhythm of the user. The method 600 may continue with sending the present circadian rhythm status to a remote computerized device 604. This may be accomplished by transmitting an indication of the status using a network communication device as discussed herein above. Moreover, the remote computerized device may be associated with an entity other than user, such as a hotel operator, a car rental company, a medical professional, or any other entity. The receiving entities make take any action responsive to the status, such as preparing a room responsive to the status, e.g. prepared for an immediate nap or provision of caffeine-containing items, determining whether the user is in an appropriate and safe status to rent and drive a car, and any other action.

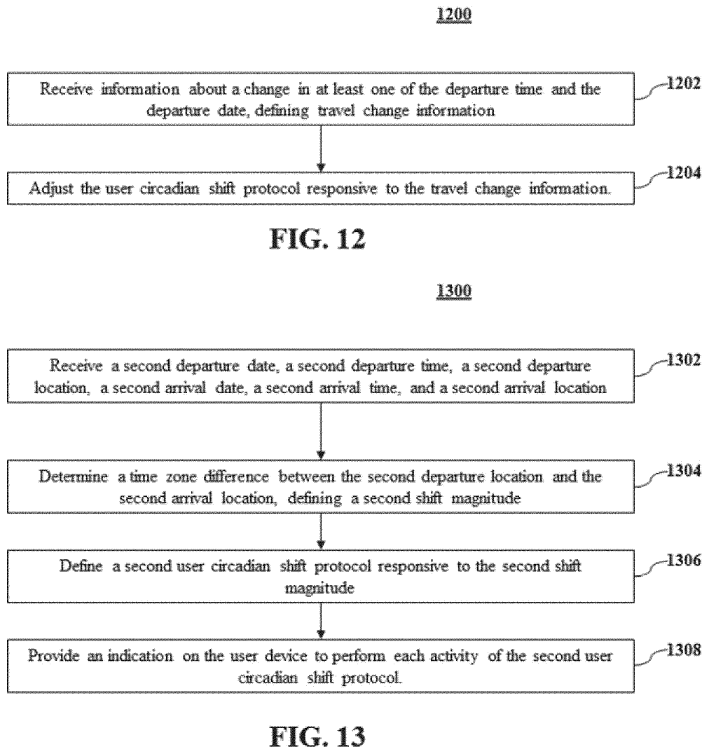

Referring now to FIG. 7, a method 700 of generating a second circadian shift protocol and modifying a circadian shift protocol responsive to a shift protocol conflict according to an embodiment of the invention is presented. In this embodiment, the protocol generated in FIG. 1 may be considered a first user circadian shift protocol. The method 700 may comprise receiving a second indication of an intended circadian shift comprising a second shift magnitude 402. The second indication may be in addition to the first indication shown at 102 in FIG. 1. The method 700 may continue with defining a second user circadian shift protocol responsive to the second shift magnitude and the user circadian phaser 704, with the user circadian phase having been received at 104 in FIG. 1. The second user circadian shift protocol may comprise circadian-shifting activities of the types described herein above. The method 700 may continue with determining whether an activity of the first user circadian shift protocol conflicts with an activity of the second user circadian shift protocol. Types of conflicts include whether the activities are coincident and unable to be performed simultaneously and/or the activities of one protocol are scheduled to occur during the performance of the other protocol and shift the circadian rhythm of the user in a manner that is inconsistent or not conducive with producing the intended circadian shift of the other protocol. Upon determining an activity of the first user circadian shift protocol conflicts with an activity of the second user circadian shift protocol, the method 700 may continue with modifying at least one of the first user circadian shift protocol and the second user circadian shift protocol to remove the conflict 708. Such modification may take the form of rescheduling one or both of the conflicting activities, changing the activity type of one or both of the conflicting activities, and combinations thereof. The method 700 may continue with providing an indication to perform each activity of the first and second user circadian shift protocols on the user device 710.

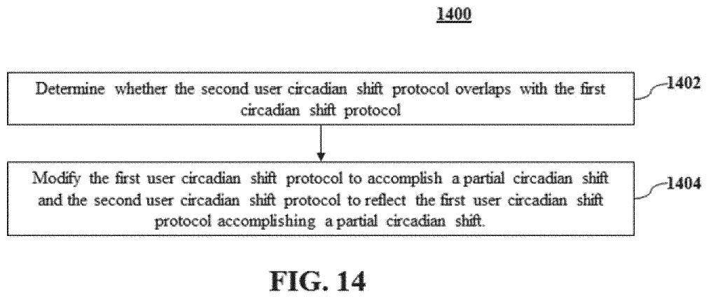

As mentioned above, the intended circadian shift may be related to a user trip, and the indication for the intended shift may be travel information. In such embodiments, the travel information may comprise a departure date, a departure time, a departure location, an arrival date, an arrival time, and an arrival location. FIGS. 8-14 are directed to such embodiments.

Referring now to FIG. 8 a method 800 of defining a circadian shift protocol comprising an activity to be performed during a travel stopover according to an embodiment of the invention is presented. The method 800 may comprise receiving travel information comprising a stopover start time and a stopover end time, with the time between the stopover start time and the stopover end time defining a stopover duration. The method 800 may continue with determining if the stopover duration is sufficient for an activity 804. The type of activity considered may affect this determination. For example, an activity involving napping would require the user having sufficient time to go to an area appropriate for napping, napping, and getting to where the user needs to be by the stopover end time. Another example is an activity involving consuming caffeinated beverage would require time for the user to go to a location that provides such beverages, time to order and consumer the beverage, and time to get to where the user need to be at the stopover end time. Activities included in this determination include, but are not limited to, light exposure or light avoidance activities, chronobiotic activities, nutritional consumption activities, physical activities, and rest activities. The method 800 may further comprise defining the user circadian shift protocol to comprise an activity during the stopover duration, defining a stopover activity.

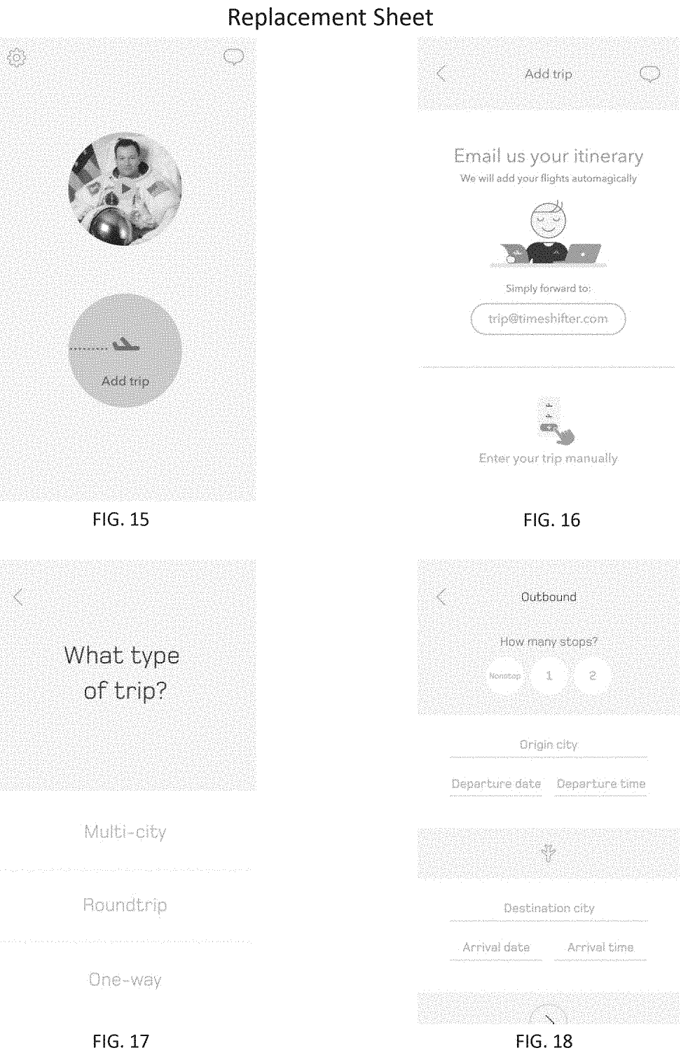

Referring now to FIG. 9, a method 900 of defining a partial circadian shift protocol according to an embodiment of the invention is presented. The method 900 may comprise determining a pre-arrival time, defined as a length of time between a present time and the arrival time on the arrival date 902. The method 902 may further comprising determining a pre-arrival required daily shift to accomplish a preferred circadian shift to a time zone associated with the arrival location 904. This may be understood as the amount the user's circadian rhythm must shift each day to accomplish the preferred circadian shift by the arrival time. In some embodiments, such a preferred circadian shift may be a complete circadian shift, i.e. the user is fully shifted to a target circadian rhythm associated with the arrival location. In some embodiments, such a complete shift may be impractical, necessitating the user to pre-shift their circadian rhythm to an extent that is not conducive to conducting their life, thereby decreasing the likelihood of compliance with the protocol. In such embodiments, the preferred circadian shift to be accomplished by the arrival time on the arrival date may be only a portion of the complete circadian shift, with the remainder of the complete circadian shift to be accomplished after arrival. It is contemplated and included within the scope of the invention to define a user circadian shift protocol for the target circadian shift to be accomplished prior to the arrival time, at the arrival time, or after the arrival time, with the method determining which is most practical and likely to be adhered to by the user.

The method 900 may further comprise determining whether the prearrival required daily shift exceeds a maximum daily shift. The maximum daily shift may be understood as a maximum amount a user can shift their circadian rhythm each day. This is commonly understood to be between 1-2 hours per day. However, it is contemplated and within the scope of the invention that the protocol may be defined assuming a maximum daily shift of three hours, accounting for the improvement that circadian-shifting activities affects on circadian-shifting capabilities of the user, when compared to non-performance of such circadian-shifting activities. In some embodiments, the user may set or adjust the maximum daily shift.

Upon determining the pre-arrival required daily shift exceeds the maximum daily shift, the method 900 may continue with modifying the user circadian shift protocol to accomplish a partial circadian shift by the arrival time on the arrival date 908. This means that only a portion of the preferred circadian shift will be accomplished at arrival time, regardless of whether the preferred circadian shift is a complete shift or a partial shift.

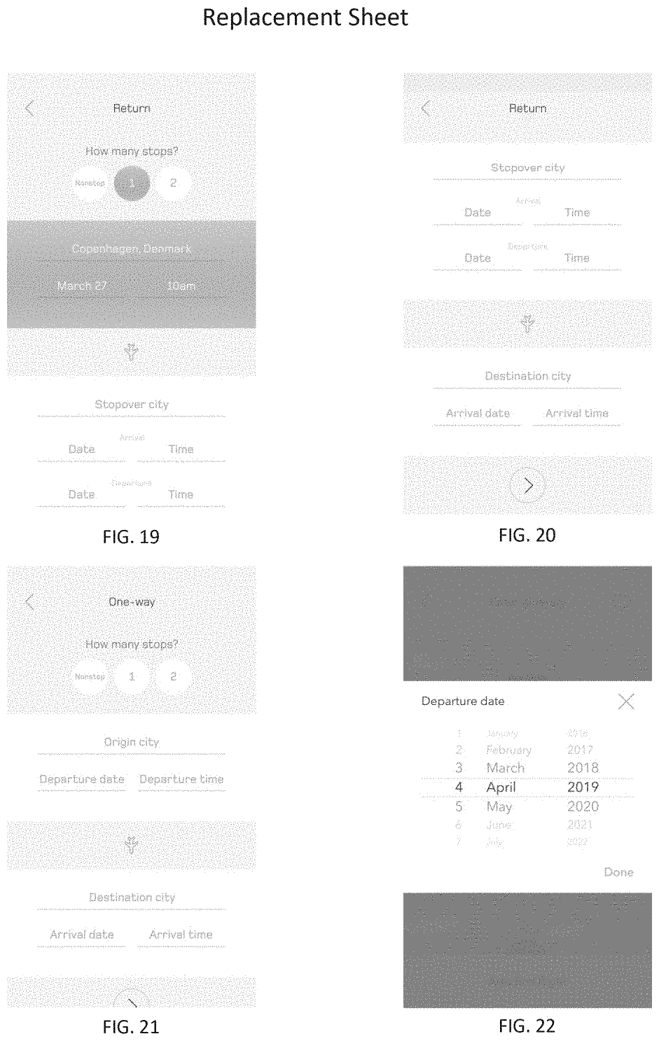

Referring now to FIG. 10, a method 1000 of defining a circadian shift protocol for a travel companion according to an embodiment of the invention is presented. The method 1000 may comprise receiving travel information for an individual other than the user, defining a companion 1002. The travel information may comprise information similar to that of the user's travel information, such as a companion departure date, a companion departure time, a companion departure location, a companion arrival date, a companion arrival time, and a companion arrival location. The method 1000 may further comprise determining a companion time zone difference between the companion departure location and the companion arrival location, defining a companion shift magnitude. In some embodiments, any or all of the companion travel information may be the same as the user travel information. In some embodiments, some of the companion travel information may be different from the user travel information. In most embodiments, there will be a connection between the user and the companion, such as a motivation for the intended circadian shift or the same arrival location, such that the user and the companion are on roughly the same circadian rhythm, accounting for their individual differences (e.g. difference in user circadian phase, chronotype, etc.).

The method 1000 may further comprise determining a companion time zone difference between the companion departure location and the companion arrival location, defining a companion shift magnitude 1004, similar to step 106 of FIG. 1. The method 1000 may further comprise receiving personal information for each of the user and the companion 1006. Such personal information may include any information about the user and companion, respectively, that may information performance of step 1008, determining the user circadian shift protocol responsive to the personal information of at least one of the user and the companion. Types of personal information include, but are not limited to, age, medical conditions, personal habits, and any other information that may relate to a greater or lesser need for the user circadian shift protocol to be adjusted to that need. As an example, if the companion is an infant, the protocol would need to reflect the ability to accommodate frequent napping and feeding, precluding long naps for the user. As another example, for a family travelling abroad, a single user may provide inputs for each member of the family that is travelling together. In another example, for a business trip including multiple adults, each adult may input their own information. In some embodiments, after each additional user information is inputted, the method may comprise determining a circadian rhythm responsive to the various inputs received. In some embodiments, some user inputs may be weighted more heavily than others, for example, a child's input may be given more or less weight than an adult's, an elderly person's input may be given more or less weight, a person with a medical requirement may be given more weight, etc. In other embodiments, a user providing an input may indicate that, when they have completed providing their input, no more users inputs will be included in the combination circadian shift protocol.

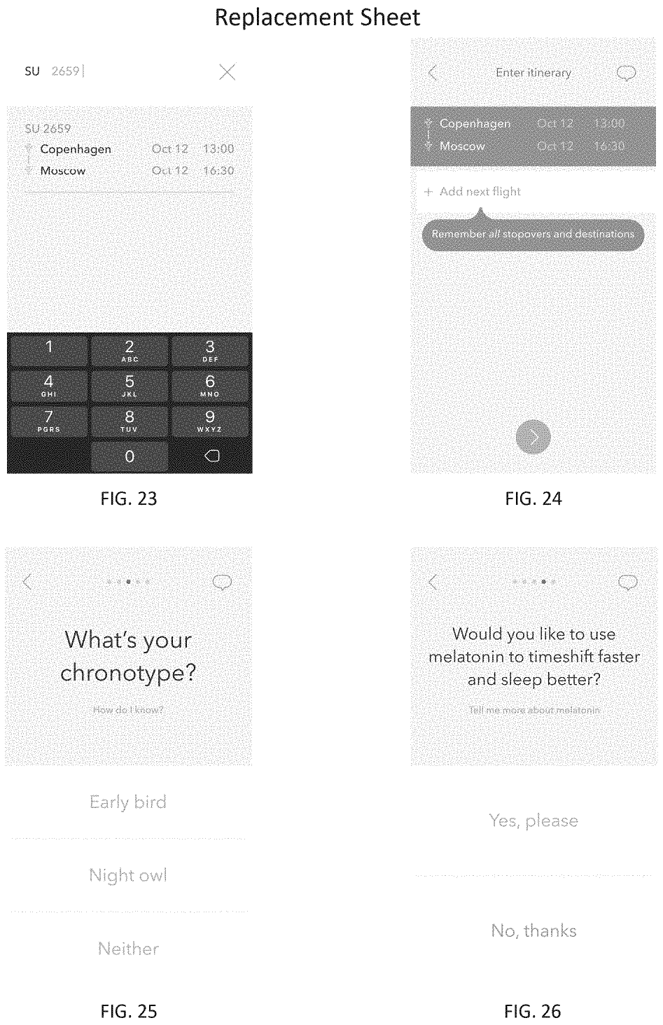

Referring now to FIG. 11, a method 1100 of defining a circadian shift protocol for two individuals according to an embodiment of the invention is presented. The method 1100 may be contemplated as being performed in conjunction with method 1000 of FIG. 1000. The method 1100 may comprise receiving an indication of a companion circadian phase, similar to the user circadian phase received in step 104 of FIG. 1. The method 1100 may further comprise defining a companion circadian shift protocol responsive to the companion shift magnitude as shown in 1004 of FIG. 10 and the companion circadian phase 1104. The companion circadian shift protocol may comprise circadian-shifting activities as described hereinabove. The method 1100 may further comprise providing an indication to perform each activity of the companion circadian shift protocol on a device, such as a companion smart phone. In some embodiments, the user device may be utilized for providing indications of the companion circadian shift protocol activities. Such embodiment may be useful where the companion requires a different circadian shift protocol than the user. For example, children may be more likely to take naps, but less likely to ingest caffeinated beverages, and therefore may have a first circadian shift protocol having greater reliance on certain activities, exposures, and substance ingestion and less reliance on others. In contrast, adults may be less likely to take naps but more likely to ingest caffeinated beverages, and therefore may have a circadian shift protocol responsive to those tendencies. Elderly adults may be relatively more likely to take naps and ingest caffeinated beverages, and therefore a circadian shift protocol responsive to these tendencies may be configured, differentiated in the recommendations made from those for children or adults.

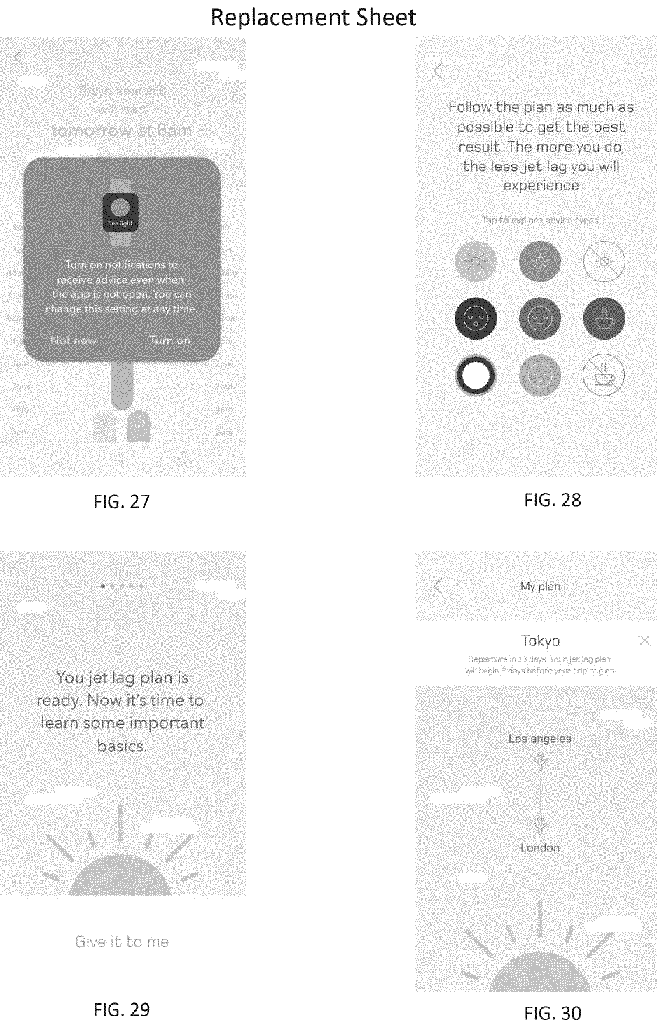

Referring now to FIG. 12, a method 1200 of modifying a circadian shift protocol responsive to a travel delay according to an embodiment of the invention is presented. The method 1200 may comprise receiving information about a change in at least one of a departure time, the departure date, the arrival time, and the arrival date defining travel change information 1202. Such a change is common with flights, trains, busses etc. being delayed or cancelled for a variety of reasons. Such an indication may be received in many ways, including, but not limited to, receiving an indication for the operator of the related travel mode, a calendar indication, or a user input. The method 1200 may further comprise adjusting the user circadian shift protocol responsive to the travel change information. Such an adjustment may include changing the time one or more activities is scheduled to be performed, adding or removing activities, changing when the complete circadian shift is accomplished, or any other aspect or element of the user circadian shift protocol.

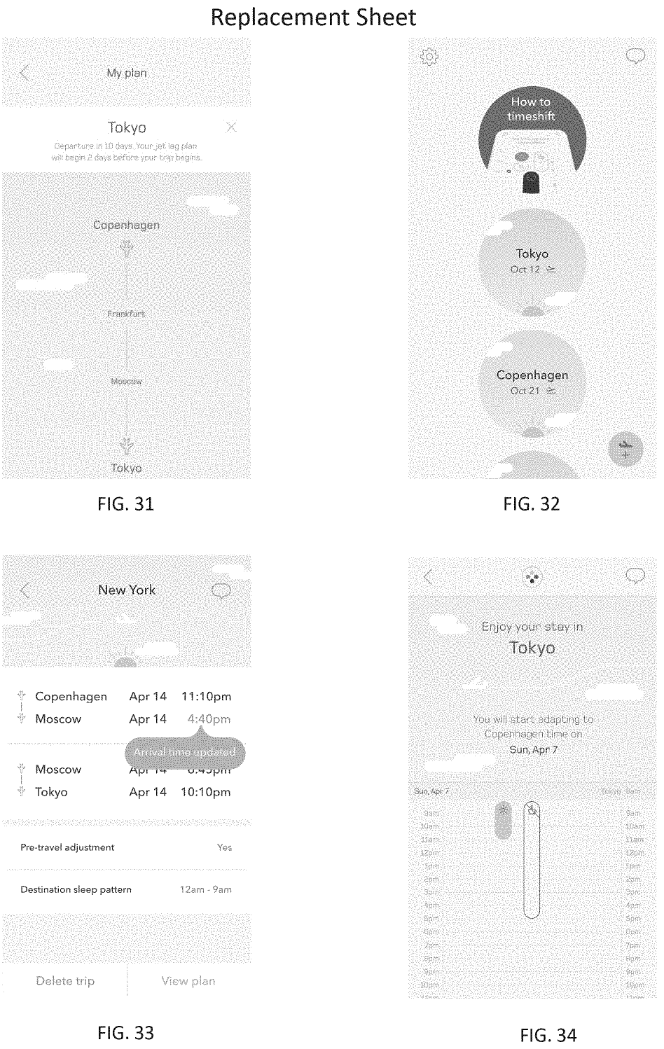

Referring now to FIG. 13, a method 1300 of defining and displaying a second circadian shift protocol subsequent to a first circadian shift protocol according to an embodiment of the invention is presented. In this embodiment, the user circadian shift protocol shown in FIG. 1 is a first user circadian shift protocol. The method 1300 may comprise receiving a second departure date, a second departure time, a second departure location, a second arrival date, a second arrival time, and a second arrival location 1302. In some embodiments, this information may be received as part of the user travel information. The method 1300 may further comprise determining a time zone difference between the second departure location and the second arrival location, defining a second shift magnitude 1304. The method 1300 may further comprise defining a second user circadian shift protocol responsive to the second shift magnitude 1306. The second user circadian shift protocol may comprise circadian-shifting activities as described hereinabove. The method 1300 may further comprise providing an indication on the user device to perform each activity of the second user circadian shift protocol 1308, as described hereinabove.

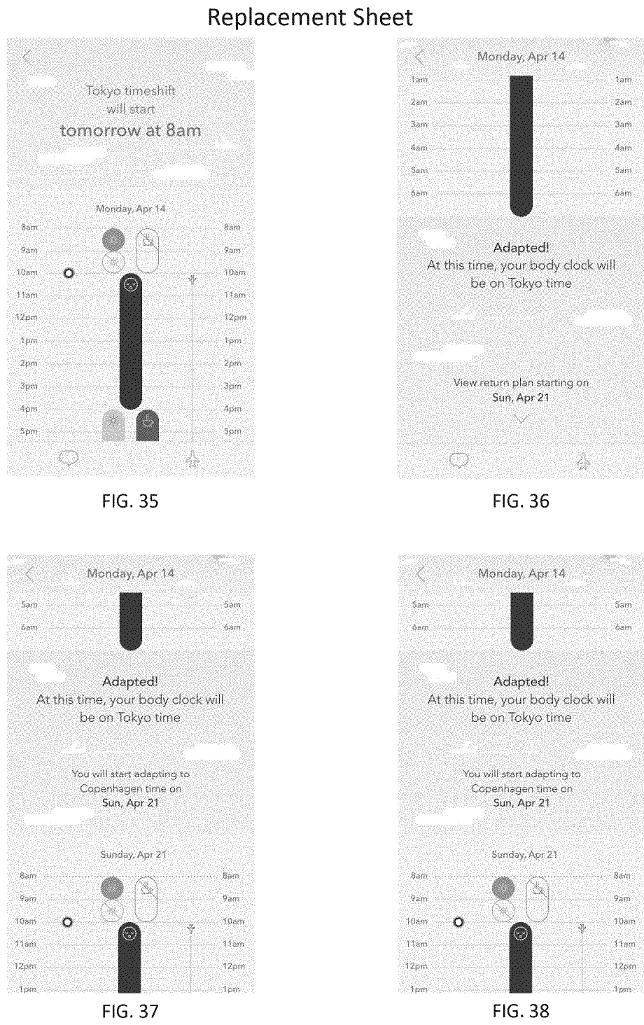

Referring now to FIG. 14, a method 1400 of modifying the circadian shift protocols of FIG. 13 responsive to a conflict there between according to an embodiment of the invention is presented. The second circadian shift protocol may be understood as an intended circadian to occur subsequent to the first circadian shift protocol, but in close proximity by time. The method 1400 may comprise determining a length of stay defined as the time between the arrival time on the arrival date and the second departure time on the second departure date 1402. The method 1400 may further comprise determining whether the second user circadian shift protocol overlaps with the first circadian shift protocol. Such an overlapping may be based on whether the second user circadian shift protocol being shifting the user's circadian rhythm prior to the first circadian shift protocol accomplishing the full shift magnitude. The method 1400 may further comprise modifying the first user circadian shift protocol to accomplish a partial circadian shift and modifying the second user circadian shift protocol to reflect the first user circadian shift protocol accomplishing only a partial circadian shift, meaning second shift magnitude may be more or less than had the first user circadian shift protocol accomplished the full shift magnitude.

In some embodiments, the user circadian shift protocol may comprise an activity scheduled to be performed after the departure time and prior to the arrival time, i.e. during the flight.

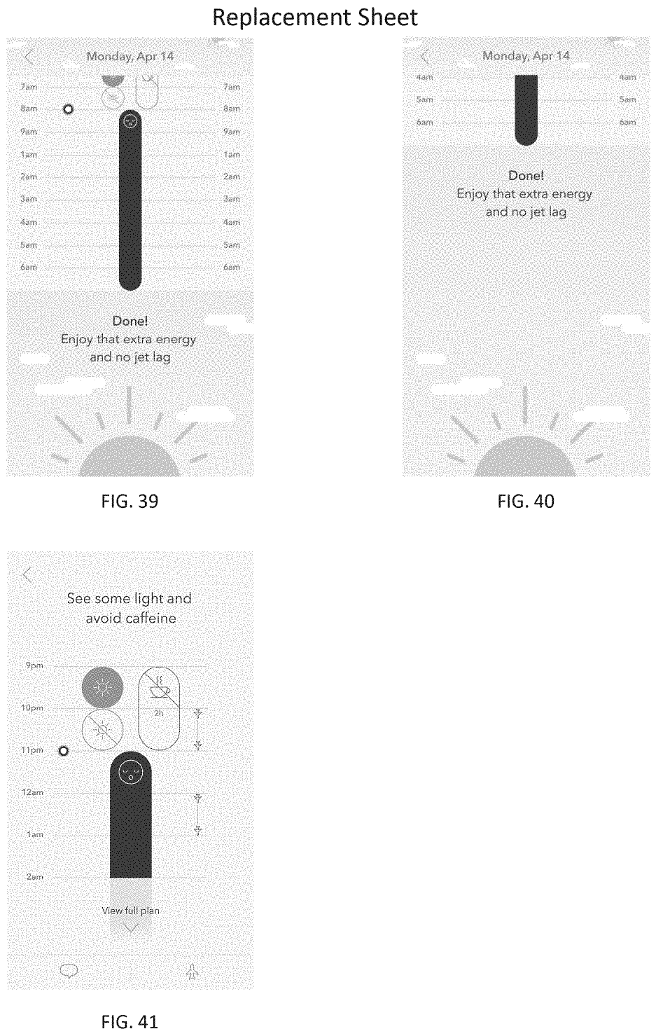

Referring now to FIGS. 15-41, displays of an exemplary user interface 1500 showing the collection of information to generate a user circadian shift protocol and providing an indication of the protocol are presented. While the displays are travel-oriented, it is contemplated and included within the scope of the invention that any motivation for accomplishing a circadian shift may have a similar and according user interface that is within the scope of the invention.

FIG. 15 depicts a display for providing the user the opportunity to provide a new indication of an intended circadian shift (a new trip). FIG. 16 provides an option for the user to provide travel information by email, with such an email being parsed to extract the relevant travel information, or to enter the travel information manually. FIGS. 17-24 present various screens for the user to enter travel information, including, but not limited to, departure date, departure time, departure location, stopover locations, stopover dates, stopover times, arrival date, arrival time, arrival locations, and flight information.

FIG. 25 presents a display to enable the user to indicate their chronotype (e.g. early bird, night owl, or neither). FIG. 26 presented a display to give the option for using chronobiotic substances (in this instance, melatonin) to facilitate the circadian shift, according to defining excluded activities as described herein above. FIG. 27 provides an indication to the user to configure an accessory device to facilitate provision of indications to perform the activities of the circadian shift protocol, in this instance, enabling notifications on a smart device, namely, a smart watch. FIG. 28 presents an overview of iconographic representation of various activities comprised by the circadian shift protocol.

FIGS. 29-32 provide displays of summaries about the circadian shift protocol responsive to the travel information, including departure location and date, stopover locations, and arrival location and date. FIG. 33 presents receipt of an updated arrival time, according with travel change information recited herein above.

FIG. 34-36 present the provision of indications on the user device to perform activities of a circadian shift protocol, particularly the iconographic representation of the activities within a calendar to indicate the timing and duration of the activities. FIGS. 37-41 present the provision of indications on the user device to perform activities of a second circadian shift protocol that succeeds the protocol shown in FIGS. 34-36.

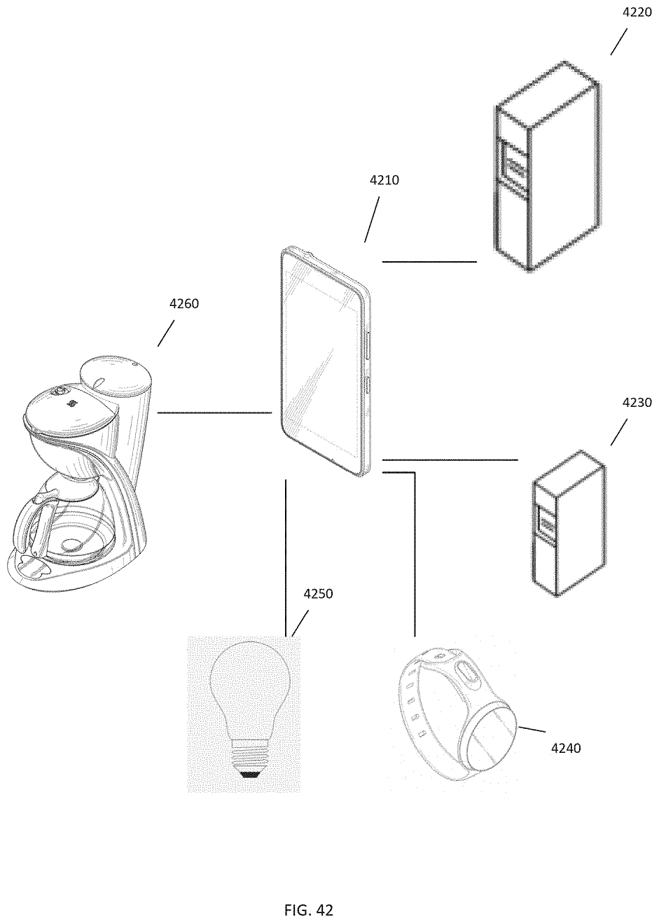

Referring now to FIG. 42, a schematic representation of connected devices that may be part of the present invention is presented. As mentioned above, the method may be implemented on a user device 4210, which may be a smart phone. In some embodiments, the method may be implemented on a server 4220 which may be positioned in communication with the user device 4210, operable to provide display information to prompt the user to enter user inputs, to received user inputs, and to provide both a calendar display of a circadian shift protocol generated responsive to the user inputs and notifications on the user device 4210 according to the circadian shift protocol. Accordingly, the method may include sending a command to a device responsive to an activity comprised by a circadian shift protocol

Additionally, at least one of the user device 4210 and the server 4220 may be positioned in communication with one or more attached devices, including, but not limited to, a third-party server 4230 that may be further attached to one or more smart devices, a smart watch 4240 that may receive and display circadian shift protocol notifications and calendar information, reformatted from how such information is delivered on the user device 4210 to account for the smaller screen or no screen, and the circumstances and context of the user. For example, a smart lamp 4250 in order to emit light having a spectral power distribution consistent with the circadian shift protocol (e.g. emitting light with greater intensity within the blue-light spectrum to simulate daylight, emitting light with less intensity in the blue-light spectrum to simulate avoiding daylight. In another example, a smart coffee maker 4260 may be communicated with to, for example, brew coffee when consuming caffeinated beverages is consistent with a circadian shift protocol, and deny brewing coffee when a circadian shift protocol recommends avoiding caffeinated beverages. In another example, a screen for crew members on an airplane display circadian shifting protocols of passengers, the passengers' needs based on their circadian shift protocols, and their compliance with the circadian shifting protocols, enabling the crew to personalize the timing of in-flight activities such as meal service, appropriate lighting, availability of coffee/tea/caffeine, melatonin, etc. consistent with such circadian shift protocols. In another example, an in-flight entertainment system display activities, exposures, and substance ingestions of a passenger's circadian shift protocol allowing the passenger to take recommended actions and maybe also use the system to order supportive products or services, including the ordering of coffee/tea/caffeine, melatonin, etc.

The circadian shifting protocol may ‘run’ the screen brightness and spectrum (e.g. reduce or increase the proportion of short wavelength blue light emitted by the screen) of any electronic device, including a computer, phone, or in-flight entertainment system on an airplane or in any other setting by altering the display characteristics of displays, altering the light emission characteristics of lighting devices. Further, the circadian shifting protocol may ‘run’ the auxiliary/overhead/seat/ambient lighting or even the window screens (either automatically pulling them up or down or filtering the intensity and spectrum of light that is transmitted) or window glass (automatically altering the filtering the intensity and spectrum of light that is transmitted). Reminders and notifications could also be presented on the screens for what to do and when based on the circadian shift protocol, taking the form of causing the display of information on a display.

Additionally, the circadian shifting protocol could ‘run’ the brightness and spectrum (e.g. reduce or increase the proportion of short wavelength blue light emitted by the screen) of hotel room lighting or even the window screens (either automatically pulling them up or down or filtering the intensity and spectrum of light that is transmitted) or window glass (automatically altering the filtering the intensity and spectrum of light that is transmitted).

If the individual wishes to follow such activities, exposures, and substance ingestion on any device, the device may offer options to support the individual in being compliant with such efforts. For example, if the user agrees to go to sleep, a movie playing on an in-flight entertainment system could be paused and an alarm clock offered to help the passenger wake up when the circadian shift protocol determined it would be optimal to wake up).

However, if the individual does wish to follow such activities, exposures, and substance ingestion, alternative suggestion may be offered to avoid impacting the circadian shift negatively (e.g. lower the brightness and a better spectrum), minimizing the negative impact when a passenger for example continues to watch a movie when they were suppose to avoid light entirely.

While the number of time zones to be crossed (and the jet lag adaptation required) will be the same for different versions of a given trip, there is variability in the degree to which the flight affects sleep patterns and the degree to which an individual can comply with the advice. In some embodiments, communication of one of the user device 4210 and the server 4220 with the third-party server 4230 may comprise identifying one or more flights that are relatively more conducive to avoiding jet lag/accomplishing a circadian shift. This may take the form of either providing a time range when it would be most conducive to the circadian shift protocol to travel, or further conducting a search of available commercial passenger flights within such a time range to provide the user with a variety of travel options that will best match the circadian shift protocol.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

That which is claimed is:

1. A method of determining and presenting a protocol for adjusting a circadian rhythm of a user comprising:
receiving an indication of an intended circadian shift comprising a shift magnitude;
receiving an indication of a user circadian phase;
defining a user circadian shift protocol responsive to the shift magnitude and the indication of the user circadian phase, the user circadian shift protocol comprising one or more activities scheduled to be performed at a certain date and time, the activities comprising at least one of:
a light exposure or light avoidance activity;
a chronobiotic activity;
a nutritional consumption activity;
a physical activity; and
a rest activity; and
providing an indication to perform each activity.

2. The method of claim 1 further comprising receiving a sleep pattern for the user, wherein the user circadian shift protocol is defined responsive to the sleep pattern.

3. The method of claim 1 further comprising receiving an activity preference indication for the user identifying an activity the user will not perform, defining an excluded activity; wherein defining the user circadian shift protocol comprises defining one or more activities excluding the excluded activity.

4. The method of claim 1 further comprising:
receiving an indication of non-performance of an activity, defining a non-performed activity; and
adjusting the user circadian shift protocol responsive to the non-performed activity.

5. The method of claim 1 further comprising:
identifying a first resting activity comprised by the user circadian shift protocol and a second resting activity comprised by the user circadian shift protocol, where no other resting activities comprised by the user circadian shift protocols are scheduled between the first resting activity and the second resting activity;
determining a length of time between the first resting activity and the second resting activity, defining a resting gap;
determining if the resting gap exceeds a maximum resting gap threshold; and
upon determining the resting gap exceeds the maximum resting gap threshold, modifying the user circadian shift protocol by at least one of:
altering at least one of the first resting activity and the second resting activity such that the resting gap is reduced to no longer exceed the maximum resting gap threshold; and
creating a new resting activity scheduled between the first resting activity and the second resting activity.

6. The method of claim 1 further comprising:
receiving an indication of an intervening user event;
determining whether the intervening user event conflicts with the performance of an activity; and
modifying the user circadian shift protocol responsive to determining the intervening user event conflicts with the performance of an activity.

7. The method of claim 1 further comprising:
identifying an activity comprised by the user circadian shift protocol that is concurrent with or in the proximate future of the current time, defining an identified action;
receiving an indication of a present location of the user;
identifying a location proximate to the present location of the user that can facilitate performance of the identified action, defining an activity location; and
providing an indication of the existence and location of the activity location.

8. The method of claim 1 further comprising:
receiving an indication of a present circadian rhythm status of the user;
receiving an indication of a present location of the user;
determining a preferred circadian rhythm status responsive to the present location of the user; and
determining if the present circadian rhythm of the user is mismatched with the preferred circadian rhythm status;
wherein the user circadian shift protocol is defined responsive to the present circadian rhythm of the user is mismatched with the preferred circadian rhythm status.

9. The method of claim 1 further comprising determining whether an advancing circadian shift protocol that advances a circadian rhythm of the user or a delaying circadian shift protocol that delays the circadian rhythm of the user is preferable, defining a preferred shifting direction; wherein the user circadian shift protocol is defined responsive to the preferred shifting direction.

10. The method of claim 1 further comprising
determining a present circadian rhythm status of the user responsive to the current time and the activities scheduled prior to the current time; and
sending the present circadian rhythm status to a remote computerized device.

11. The method of claim 1 wherein the user circadian shift protocol is a first user circadian shift protocol, the method further comprising
receiving a second indication of an intended circadian shift comprising a second shift magnitude;
defining a second user circadian shift protocol responsive to the second shift magnitude, the second user circadian shift protocol comprising one or more activities scheduled to be performed at a certain date and time, the activities comprising at least one of:
a light exposure or light avoidance activity;
a chronobiotic activity;
a nutritional consumption activity;
a physical activity; and
a rest activity; and
determining whether an activity of the first user circadian shift protocol conflicts with an activity of the second user circadian shift protocol; and
upon determining an activity of the first user circadian shift protocol conflicts with an activity of the second user circadian shift protocol; modifying at least one of the first user circadian shift protocol and the second user circadian shift protocol to remove the conflict; and
providing an indication to perform each activity of each of the first user circadian protocol and the second user circadian protocol.

12. The method of claim 1 further comprising sending a command to a device responsive to an activity comprised by the user circadian shift protocol.

13. The method of claim 12 wherein the command is selected from the group consisting of altering display characteristics of a display, causing a display of information on a display, altering light emission characteristics of a lighting device, altering a status of a window-related device to alter ambient light conditions, initiating production of a beverage comprising a stimulant, and transmitting a message to a person regarding provision of at least one of a chronobitotic and nutrition.

14. The method of claim 1 wherein the indication of the intended circadian shift is related to travel information for a user trip, the travel information comprising a departure date, a departure time, a departure location, an arrival date, an arrival time, and an arrival location; and wherein a time zone difference between the departure location and the arrival location defines the shift magnitude.

15. The method of claim 14 wherein the travel information further comprises a stopover start time and a stopover end time, the time between the stopover start time and the stopover end time defines a stopover duration, the method further comprising:
determining if the stopover duration is sufficient for an activity; and
upon determining the stopover duration is sufficient for an activity, defining the user circadian shift protocol to comprise an activity during the stopover duration, defining a stopover activity;
wherein the stopover activity is selected from the activities consisting of a light exposure or light avoidance activity, a chronobiotic activity, a nutritional consumption activity, a physical activity, and a rest activity.

16. The method of claim 14 wherein the user circadian shift protocol comprises a maximum daily shift, the method further comprising:
determining a pre-arrival time defined as a length of time between a present time and the arrival time on the arrival date;
determining a pre-arrival required daily shift to accomplish a complete circadian shift to a time zone associated with the arrival location;
determining whether the pre-arrival required daily shift exceeds the maximum daily shift; and
upon determining the pre-arrival required daily shift exceeds the maximum daily shift, modifying the user circadian shift protocol to accomplish a partial circadian shift by the arrival time on the arrival date.

17. The method of claim 14 further comprising:
receiving travel information for an individual other than the user, defining a companion, comprising a companion departure date, a companion departure time, a companion departure location, a companion arrival date, a companion arrival time, and a companion arrival location;
determining a companion time zone difference between the companion departure location and the companion arrival location, defining a companion shift magnitude; and
receiving personal information for each of the user and the companion;
wherein the user circadian shift protocol is determined responsive to the personal information of at least one of the user and the companion.

18. The method of claim 17 further comprising:
receiving an indication of a companion circadian phase;
defining a companion circadian shift protocol responsive to the shift magnitude and the indication of the companion circadian phase, the circadian shift protocol comprising one or more activities, the activities comprising at least one of:
a light exposure or light avoidance activity;
a chronobiotic activity;
a nutritional consumption activity;
a physical activity; and
a rest activity; and
providing an indication to perform each activity of the companion circadian shift protocol.

19. The method of claim 14 further comprising:
receiving information about a change in at least one of the departure time and the departure date, defining travel change information; and
adjusting the user circadian shift protocol responsive to the travel change information.

20. The method of claim 14 wherein the user circadian shift protocol is a first user circadian shift protocol, and wherein the travel information further comprises a second departure date, a second departure time, a second departure location, a second arrival date, a second arrival time, and a second arrival location, the method further comprising:
determining a time zone difference between the second departure location and the second arrival location, defining a second shift magnitude;
defining a second user circadian shift protocol responsive to the second shift magnitude, the second user circadian shift protocol comprising one or more second activities, the second activities comprising at least one of:
a light exposure or light avoidance activity;
a chronobiotic activity;
a nutritional consumption activity;
a physical activity; and
a rest activity; and
providing an indication to perform each activity of the second user circadian shift protocol.

21. The method of claim 20 further comprising:
determining whether the second user circadian shift protocol overlaps with the first circadian shift protocol;
upon determining the second user circadian shift protocol overlaps with the first circadian shift protocol, modifying the first user circadian shift protocol to accomplish a partial circadian shift and modifying the second user circadian shift protocol to reflect the first user circadian shift protocol accomplishing a partial circadian shift.

22. The method of claim 14 wherein the user circadian shift protocol comprises an activity scheduled to be performed after the departure time and before the arrival time.

23. A method of determining and presenting a protocol for adjusting a circadian rhythm of a user comprising:
receiving an indication of an intended circadian shift comprising a shift magnitude;
receiving an indication of a user circadian phase;
receiving an activity preference indication from the user identifying an activity the user will not perform, defining an excluded activity;
receiving an indication of a present location of the user;
determining a preferred circadian phase status responsive to the present location of the user; and
determining if the present circadian phase status of the user is mismatched with the preferred circadian phase status;
determining whether an advancing circadian shift protocol that advances a circadian rhythm of the user or a delaying circadian shift protocol that delays the circadian rhythm of the user is preferable, defining a preferred shifting direction;
defining a user circadian shift protocol responsive to the shift magnitude, the circadian phase, the preferred shifting direction, and if the present circadian phase of the user is mismatched with the preferred circadian phase status, the user circadian shift protocol comprising one or more activities scheduled to be performed at a certain date and time excluding the excluded activity, the activities comprising a first rest activity, a second rest activity, and at least one of:
a light exposure or light avoidance activity;
a chronobiotic activity;
a nutritional consumption activity; and
a physical activity;
providing an indication to perform each activity of the user circadian shift protocol; and
sending a command to a device responsive to an activity comprised by the user circadian shift protocol;
wherein a length of time between the first resting activity and the second resting activity do not exceed a maximum resting gap.

24. A method of determining and presenting a protocol for adjusting a circadian rhythm of a user comprising:
receiving a travel information related to an upcoming user trip comprising a departure date, a departure time, a departure location, an arrival date, an arrival time, and an arrival location, a stopover start time and a stopover end time, wherein a time zone difference between the departure location and the arrival location defines a shift magnitude, and wherein the time between the stopover start time and the stopover end time defines a stopover duration;
determining if the stopover duration is sufficient for an activity;
receiving an indication of a user circadian phase;
defining a user circadian shift protocol responsive to the shift magnitude, the circadian phase, and if the stopover duration is sufficient for an activity, the user circadian shift protocol comprising one or more activities scheduled to be performed at a certain date and time, the activities comprising at least one of:
a light exposure or light avoidance activity;
a chronobiotic activity;
a nutritional consumption activity;
a physical activity; and
a rest activity; and
providing an indication to perform each activity of the user circadian shift protocol;
wherein the user circadian shift protocol comprises an activity during the stopover duration if the stopover duration is sufficient for an activity selected from the activities consisting of a light exposure or light avoidance activity, a chronobiotic activity, a nutritional consumption activity, a physical activity, and a rest activity.

25. A method of determining and presenting a protocol for adjusting a circadian rhythm of a user comprising:
receiving an indication of an intended circadian shift comprising a shift magnitude;
defining a user circadian shift protocol responsive to the shift magnitude, the user circadian shift protocol comprising one or more activities scheduled to be performed at a certain date and time, the activities comprising at least one of:
a light exposure or light avoidance activity;
a chronobiotic activity;
a nutritional consumption activity;
a physical activity; and
a rest activity; and
providing an indication to perform each activity.
